(19) United States
(12) Patent Application Publication
(10) Pub. No.: US 2007/0110675 A1
(43) Pub. Date: May 17, 2007

(54) Method for predicting the developmetn of allergic disease

(75) Inventor: Patrick Holt, Nedlands (AU)

Correspondence Address: EDWARDS & ANGELL, LLP, P.O. BOX 55874, BOSTON, MA, 02205, US

(73) Assignee: Advanced Diagnostic Systems PTY LTD, Subiaco (AU)

(21) Appl. No.: 10/558,776

(22) PCT Filed: May 31, 2004
(86) PCT No.: PCT/AU04/00730

§ 371 Date: Nov. 8, 2006

(30) Foreign Application Priority Data
Jun. 3, 2003 (AU) 2003902792

(51) Int. Cl. A61K 49/00

(52) U.S. Cl. 424/9.81

(57) ABSTRACT

The present invention relates to a method for improving the effectiveness of immunotherapy. In particular, the present invention relates to method for predicting the development of an allergic disease in a subject, comprising providing a biological sample; stimulating said sample with an allergen in vitro to produce one or more cytokine(s); determining which cytokines are present; and correlating development of an allergic disease with the presence or absence of particular cytokines.

### FIELD OF THE INVENTION

The present invention relates to a method for improving the effectiveness of immunotherapy. In particular, the present invention relates to a method of predicting the development of allergic disease in subject.

### BACKGROUND OF THE INVENTION

Allergies represent one of the most common and well characterised immune disorders in humans, affecting roughly 20 percent of all individuals in the United States. Allergic reactions are generally immune reactions that are initiated by IgE-dependent stimulation of tissue mast cells and related effector molecules (eg., basophils). Binding events between cell surface bound IgE molecules and antigen results in rapid release of biological response modifiers which bring about increased vascular permeability. vasodilation, smooth muscle contraction and local inflammation. This sequence of events is termed immediate hypersensitivity and begins rapidly, usually within minutes of exposure in a sensitised individual to antigen. In its most severe systemic form, anaphylaxis, such immediate hypersensitivity can bring about asphyxiation, produce cardiovascular collapse, and even result in death. Individuals that are prone to strong immediate hypersensitivity responses are referred to as “atopic” and are said to suffer from “allergies.” Clinical manifestations of atopy include hay fever (rhinitis), asthma, urticaria (hives), skin irritation (eg., chronic eczema), and related conditions.

A number of clinical test procedures for assessing allergies have been described and are known in the art. See generally American College of Physicians, “Allergy Testing,” Ann. Intern. Med. (1989) 110:317-320; Bousquet, J. (1988) “In Vivo Methods for Study of Allergy: Skin Tests, Techniques, and Interpretation,” Allergy, Principals and Practice, 3.sup.rd ed., Middleton et al., eds., CV Mosby Co., St. Louis, Mo., pp. 419-436; and Van Arsdel et al. (1989) Ann. Intern. Med. 110:304-312. These so-called “skin prick tests” or “scratch tests” involve introduction of antigens into the epidermis via skin prick or scratch, or introduction into the dermis via intracutaneous (intradermal) injection. An immediate wheal and flare reaction at the site of introduction (the classic atopic reaction) indicates antibody-mediated (IgE) hypersensitivity to the test antigen. More particularly, when a sensitised individual is challenged by an appropriate antigen in a skin or scratch test, the site of introduction becomes red from local vasodilation. In a second phase of the reaction, soft swelling occurs (the wheal) and, in a third phase, blood vessels at the margins of the wheal dilate and become engorged with red blood cells, producing a characteristic erythemic rim (the flare). The full wheal and flare reaction usually appears within 10 to 15 minutes of antigen administration, and generally subsides within about an hour. A wheal of sufficient size with accompanying flare represents a positive test for allergy against the antigen.

However, while these simple immunological tests are available they often only provide information about an individuals current immunological status. There are no current methods of predicting the immune status of an individual.

The inventors have previously shown that the T-lymphoid system in humans engages in active surveillance for environmental allergens throughout life, and that it is the nature of (as opposed to the mere presence of) allergen-specific T-cell responses in individuals that determines whether they express the allergic (atopic) or immunologically normal (non-responder) phenotype.

The inventors have also recognised that selection of the appropriate T-cell population is an antigen-driven process which occurs during the early stages of immune responses in the naive (unsensitised) host. If selection favours the growth of allergen-specific T-cells of the T-helper-1-like (TH-1)-like phenotype low-grade non-pathogenic IgG and IgA responses ensue, whereas the emergence of TH-2-like cells can lead to IgE production and eosinophilia and ultimately atopic disease. Additionally, TH-1-like cytokines actively suppress the expansion of TH-2-like clones, and hence a dominant, stable TH-1-like response to an allergen is proposed to be actively protective against the development of TH-2-like dependent allergic disease. With respect to T-cell responses to ubiquitous environmental allergens, the inventor's review of the recent pediatric literature has identified early childhood as the life period during which this selection normally occurs, and shows that the process can take several years to complete. Once the significance of the selection is appreciated, sufficient information is already known of how this natural selection process operates to contemplate controlling it in vivo, via deliberate administration of allergen(s) in a form adapted to preferentially stimulate the development of host-protective TH-1-like immunity.

In the inventors previous application, U.S. Pat. No. 6,333,038 (“U.S. Pat. No. 6,333,038”), incorporated herein in its entirety by reference, methods and compositions for the prophylaxis of allergic disease, and in particular to allergic disease triggered by environmental antigens or allergens are described. U.S. Pat. No. 6,333,038, describes in detail the novel and unexpected mechanism for inducing protective immunity discussed briefly above. Since the filing of U.S. Pat. No. 6,333,038 the inventors have been following the progression of allergy in a cohort of children in an endeavour to increase the knowledge surrounding the mechanism for inducing protective immunity.

### SUMMARY OF THE INVENTION

Inventors have now shown that by using in vitro allergen-specific T-cell stimulation and measurement of cytokine responses, subjects can be identified that will subsequently develop allergy.

Accordingly, in a first aspect, the invention provides a method for predicting the development of an allergic disease in a subject, comprising:
a). providing a biological sample;
b). stimulating said sample with an allergen in vitro to produce one or more cytokine(s);
c). determining which cytokines are present; and
d). correlating development of an allergic disease with the presence or absence of particular cytokines.

Persons skilled in the art will appreciate that the techniques disclosed herein may be used on any type of biological sample. However, it is preferable that the biological sample is blood or a blood component. Most preferably, the biological sample is peripheral blood leucocytes.

Suitable allergens will be known to the person skilled in the art. Preferably the allergen is an environmental antigen, and may be used either singly or as a combination of two or more such allergens. The allergen may be in its naturally-occurring form. Alternatively the allergen may be a protein prepared using recombinant DNA technology, or may be a synthetic peptide.

The allergen may be in purified form or may be impure or partially purified. The allergen may represent either the whole allergen molecule, or may be a part thereof, for example including one or more epitopes.

Allergens contemplated to be suitable for use in the invention include those from house dust mite, animal danders such as cat, dog or bird dander, cockroach, grass pollens such as those from ryegrass or alternaria, tree pollens such as those from birch or cedar, feathers and moulds.

The most suitable allergens will depend on the geographical location. For example, birch and cedar pollens are a major cause of allergies in northern Europe and Japan, but are of minor importance in Australia.

The cytokines produced will depended upon the type of cells present. For example, if selection favours the growth of Th-2 cells then the possible cytokines present might include IL-4, IL-5, IL-10 and/or IL-13, which are produced by Th-2 cells. If the subject is likely to develop allergy with time then the cytokines will likely be Th2 cytokines.

In one aspect of the invention the method of predicting the development of an allergic disease in a subject, comprises the step of skin pricking a subject and administering either a Th-1 or Th-2 specific allergen. If local inflammation at the skin prick site takes place then the subject is hypersensitive to the Th-1 or Th-2 allergen. In other words, a positive skin prick test to an allergen is a surrogate for a positive serum IgE-specific antibody test. This is because during the development of allergic disease, an allergen-specific Th2 secretory response must precede B-cell production of IgE antibody, which relies on the presence of Th2 cytokines.

Accordingly, in a second aspect the invention provides a method of predicting the development of an allergic disease in a subject, comprising:
a). skin prick testing said subject with an allergen capable of inducing Th2 cytokine secretion;
b). determining level of local inflammation; and
c). correlating level of local inflammation with the development of an allergic disease.

The subject is a mammalian subject such as dogs, cats, livestock, primates and horses as well as humans. Preferably, the subject is a human subject. More preferably, the subject is a human subject below the age of about 5. Most preferably, the human subject is below the age of 2 years.

The foregoing and other aspects of the present invention are explained in greater detail in the specification below.

### BRIEF DESCRIPTION OF THE FIGURES

FIG. 1 shows the results of HDM-specific IL-5 and IL-13 responses (as mean+SEM) in PBMC at 1 year of age for those who were subsequently SPT− versus the SPT+allergic children.

### DETAILED DESCRIPTION OF THE INVENTION

Before describing the present invention in detail, it is to be understood that this invention is not limited to particularly exemplified diagnostic antigens, allergens or process parameters as such may, of course, vary. It is also to be understood that the terminology used herein is for the purpose of describing particular embodiments of the invention only, and is not intended to be limiting.

All publications, patents and patent applications cited herein, whether supra or infra, are hereby incorporated by reference in their entirety. However, publications mentioned herein are cited for the purpose of describing and disclosing the protocols, reagents and vectors which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional immunological techniques, chemistry and pharmacology within the skill of the art. Such techniques are well known to the skilled worker, and are explained fully in the literature. See, eg., Coligan, Dunn, Ploegh, Speicher and Wingfield “Current protocols in Protein Science” (1999) Volume I and II (John Wiley & Sons Inc.); and Bailey, J. E. and Ollis, D. F., Biochemical Engineering Fundamentals, McGraw-Hill Book Company, NY, 1986.

Before the present methods are described, it is understood that this invention is not limited to the particular materials and methods described, as these may vary. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “an,” and “the” include plural reference unless the context clearly dictates otherwise. Thus, for example, a reference to “a protein” includes a plurality of such proteins, and a reference to “an allergen” is a reference to one or more allergens, and so forth. Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

Definitions

Unless defined otherwise, all technical and scientific terms used herein have the same meaning as commonly understood by one of ordinary skill in the art to which the invention pertains. The following terms are intended to be defined as indicated below.

An “immunological response” or “immune response” against a selected agent or a composition of interest is the development in an individual of a humoral and/or a cellular immune response to molecules (eg., antigens or allergens) present in the agent or composition of interest. For purposes of the present invention, a “humoral immune response” refers to an immune response mediated by antibody molecules, while a “cellular immune response” is one mediated by T-lymphocytes and/or other white blood cells.

An individual previously exposed (sensitized) to a particular immunologic agent (eg., an antigen or allergen) will typically exhibit a detectable immunological response upon subsequent encounters with that agent. When the subsequent encounter takes place in skin tissue, the detectable immunological response can entail a localized skin immune reactions at the point of encounter which is due to local injury to normal self tissue brought about by components of the immunological response directed against the agent. There are generally four major types of localized skin immune reactions that can be classified based on the principal pathogenic mechanism responsible for the localized skin cell/tissue injury. The first type of localized skin immune reaction is termed “Type I immediate hypersensitivity” which is caused by IgE antibodies, mast cells and their mediators (vasoactive amines, lipid mediators and cytokines). Type I hypersensitivity reactions are generally directed against allergens such as plants, chemicals, materials, and foods. A second type of hypersensitivity, also caused by antibodies, is termed “Type II antibody-mediated hypersensitivity.” In this case, antibodies other than IgE (i.e., IgM and IgG) can cause tissue injury by recruiting and activating leukocytes (neutrophils, macrophages) and by activating the complement system. The third type of skin reaction, “Type III immune complex-mediated hypersensitivity” involves tissue damage brought about by immune complexes of circulating antigens and IgM or IgG antibodies which activate complement and recruit and activate leukocytes. Type II and III hypersensitivity reactions are generally directed against antigens associated with infectious pathogens, cancers, autoimmune disorders, or incompatible cells such as blood cells (eg., blood transfusion or Rh incompatibility) or tissue cells (eg., transplanted organs or tissue). The fourth type of skin reaction, termed “Type IV T cell-mediated hypersensitivity” involves local skin tissue damage brought about by CD4⁺ T cells, activated macrophages and cytokines (delayed type hypersensitivity or DTH) or CD8⁺ T cells and cytokines (T cell-mediated cytolysis). Type IV hypersensitivity reactions are also generally directed against antigens associated with infectious pathogens, cancers or autoimmune disorders, as well as agents involved in contact dermatitis conditions. For the purposes of the invention, reference to “a localized skin immune reaction” encompasses any one of the four major types of hypersensitivity reactions unless expressly stated otherwise.

In the practice of the invention, the presence or absence of localized skin immune reactions can be readily assessed according to known clinical procedures. For example, such skin reactions can be assessed qualitatively, eg., visually. A Type I skin reaction is usually in the form of urticaria and a wheal which appears within minutes (the early phase reaction) after antigen challenge, developing into inflammation within about 6-24 hours (the late phase reaction). The pathogenic damage associated with this cutaneous reaction is generally characterised by oedema, vascular dilation, and local smooth muscle contraction. A Type II skin reaction is typically in the form of local induration and erythema occurring over a period of 4-48 hours after antigen challenge. A Type III skin reaction is usually in the form of an Arthus reaction (local cutaneous vasculitis) which occurs within about 2-6 hours of the antigen challenge. Pathogenic damage includes necrotising vasculitis. Type IV skin reactions usually occur within about 24-48 hours of antigen challenge, and are typified by induration and/or erythema. Pathogenic tissue damage includes perivascular cellular infiltrates and oedema. All four of these major types of skin reactions can, of course, also be assessed quantitatively using calipers, ultrasound, chromameter and laser-Doppler techniques well known to those skilled in the art. Accordingly, the present invention is not limited by the manner in which the localised skin immune response is assessed or otherwise characterised.

An antigen refers to any immunogeneic moiety or agent, generally a macromolecule, which can elicit an immunological response in an individual. The term may be used to refer to an individual macromolecule or to a homogeneous or heterogeneous population of antigenic macromolecules. As used herein, “antigen” is generally used to refer to a hapten, an organic or inorganic substance, or a protein molecule or portion thereof which contains one or more epitopes. For purposes of the present invention, antigens can be obtained or derived from any known virus, bacteria, parasite or fungal pathogen, a plant, or from man-made or naturally occurring inorganic or organic material. The term also intends any of the various tumour-specific antigens and antigens associated with autoimmune diseases. Furthermore, for purposes of the present invention, an “antigen” includes a protein having modifications, such as deletions, additions and substitutions (generally conservative in nature) to the native sequence, so long as the protein maintains sufficient immunogenicity. These modifications may be deliberate, for example through site-directed mutagenesis, or may be accidental, such as through mutations of hosts which produce the antigens.

In various aspects of the invention, the antigen contains one or more T cell epitopes. A “T cell epitope” refers generally to those features of a peptide structure which are capable of inducing a T cell response. In this regard, it is accepted in the art that T cell epitopes comprise linear peptide determinants that assume extended conformations within the peptide-binding cleft of MHC molecules, (Unanue et al. (1987) Science 236:551-557). As used herein, a T cell epitope is generally a peptide having at least about 3-5 amino acid residues, and preferably at least 5-10 or more amino acid residues. The ability of a particular antigen to stimulate a cell-mediated immunological response may be determined by a number of well-known assays, such as by lymphoproliferation (lymphocyte activation) assays, CTL cytotoxic cell assays, or by assaying for T-lymphocytes specific for the antigen in a sensitized subject. See, eg., Erickson et al. (1993) J. Immunol. 151:4189-4199; and Doe et al. (1994) Eur. J. Immunol. 24:2369-2376

In other aspects of the invention, the antigen contains one or more B cell epitopes. A “B cell epitope” generally refers to the site on an antigen to which a specific antibody molecule binds. The identification of epitopes which are able to elicit an antibody response is readily accomplished using techniques well known in the art. See, eg., Geysen et al. (1984) Proc. Natl. Acad. Sci. USA 81:3998-4002 (general method of rapidly synthesising peptides to determine the location of immunogenic epitopes in a given antigen); U.S. Pat. No. 4,708,871 (procedures for identifying and chemically synthesising epitopes of antigens); and Geysen et al. (1986) Molecular Immunology 23:709-715 (technique for identifying peptides with high affinity for a given antibody).

The term “allergen” as used herein refers to any foreign antigen which stimulates allergic-type immune responses, characterised by activation of TH-2 lymphocytes and production of specific IgE antibody.

The term “environmental allergen” means any allergen found in the environment. These allergens are usually, but not necessarily, naturally occurring.

As used herein, the term “anergy” refers to either a diminished immune reaction, or the absence of an immune reaction to an antigen as revealed by the lack of an appropriate immune response (as detectable by a reduced localized skin immune reaction to a diagnostic antigen or allergen as administered according to the present invention). Anergy can further entail a reversible antiproliferative state which results in decreased responsiveness of an immune cell or cells to an antigen.

The term “subject” or “individual” are used interchangeably herein to refer to any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly.

Thus, provided is the treatment of mammals such as humans, as well as those mammals of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, eg., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

In one embodiment, the subject is a human child between 3 months and 7 years old, but is also applicable to individuals older than 7 years. Preferably the child is not less than 6 months old, more preferably not less than 9 months old.

Because in early childhood most individuals will not yet have been exposed to sensitisation by environmental allergens, it is considered that this period provides the optimum opportunity to predict the likely onset of allergy.

The term “sensitisation” refers to the effect of “priming” populations of T-cells to respond specifically to subsequent challenge with the priming antigen or allergen. In the context of this specification, this refers to the priming of allergen-specific TH-2 cells. The term “desensitisation” refers to the therapeutic administration of allergen, or a derivative thereof, to allergen-reactive allergic individuals, with the aim of selective suppression of the activity of allergen-specific T-cells, in particular TH-2 cells, and/or other cell types recruited into the allergen-specific immune or allergic response.

General Methods

The present invention relates to a method of predicting the development of an allergic disease in a subject.

In principle, inventors believe that by using in vitro allergen-specific T-cell stimulation and measurement of cytokine responses, children can be identified who will subsequently manifest allergy. The underlying principles are as follows:

1). The immunological basis for allergy is possession of T-helper cell which secrete Th2 cytokines;

2). These T-cells drive the differentiation of B-cells to produce the allergen specific IgE which is responsible for “immediate hypersensitivity” reactions ie. the early phase of allergic responses in tissues;

3). Detection of this hypersensitivity takes the form of a positive skin prick test (“SPT”) i.e. local inflammation at a skin site after injection of specific allergen such as house dust mite (“HDM”)—a positive SPT to HDM is thus a surrogate for positive serum IgE-specific antibody;

4). During the development of allergic disease, development of allergen-specific Th2 secretory responses must (by definition) precede B-cell production of IgE antibody, which relies on the presence of Th2 cytokines.

In one embodiment, individuals before the age of 2 year are skin prick tested using Th2 allergens such as HDM. As these children have yet to develop their immune system; however, if the child shows a positive Type I skin reaction within 10 minutes then it is likely that this individual will develop an allergy with time to HDM.

Throughout the specification, unless the context requires otherwise, the word “comprise” or variations such as “comprises” or “comprising”, will be understood to imply the inclusion of a stated integer or group of integers but not the exclusion of any other integer or group of integers.

The invention will now be further described by way of reference only to the following non-limiting examples. It should be understood, however, that the examples following are illustrative only, and should not be taken in any way as a restriction on the generality of the invention described above. In particular, while the invention is described in detail in relation to the identification of specific Th2 cytokines, it will be clearly understood that the findings herein are not limited to these cytokines. For example, other Th-2 cytokines may be tested.

### EXAMPLE 1

### Assessment of Children for Possible Allergy

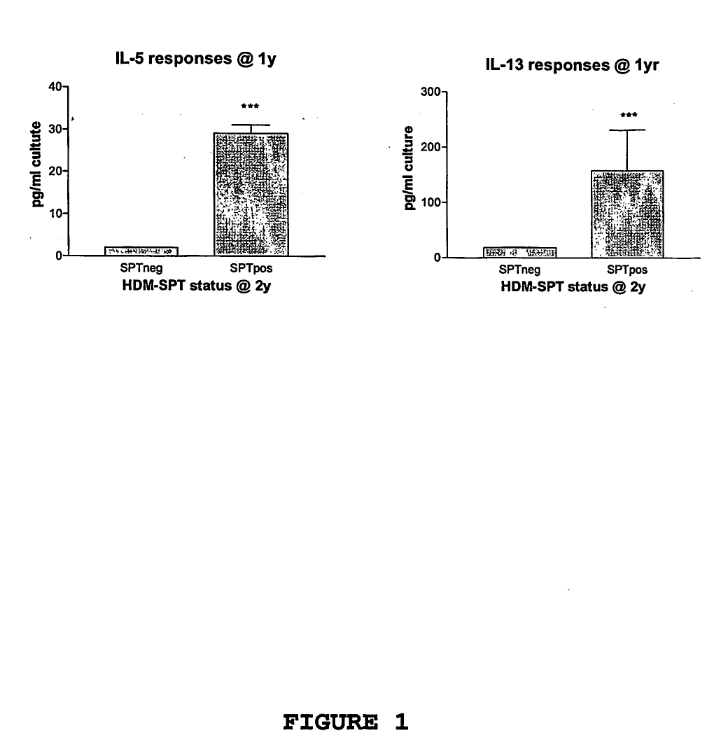

223 Infants were bled at 1 yr age and their PBMC cryopreserved. At this age clinical sensitisation to inhalant allergens such as HDM is rare (usually <5%). At 2 yrs age allergic sensitisation to HDM was assessed by skin prick test (SPT), and in parallel the cryopreserved peripheral blood mononuclear cells (PBMC) were thawed and stimulated in vitro with HDM, and cytokine production assessed. 21% of the children were HDM-SPT+at 2 years. Results shown in FIG. 1 demonstrate that HDM-specific IL-5 and IL-13 responses (as mean+SEM) for those who were SPT− were statistically significantly lower than the allergic SPT+children (***: p<0.000 by Mann Whitney test).

1. A method for predicting the development of an allergic disease in a subject, comprising:
a). providing a biological sample;
b). stimulating said sample with an allergen in vitro to produce one or more cytokine(s);
c). determining which cytokines are present; and
d). correlating development of an allergic disease with the presence or absence of particular cytokines.

2. A method according to claim 1, wherein the biological sample is blood or a blood component.

3. A method according to claim 1 or claim 2, wherein the biological sample is peripheral blood leucocytes.

4. A method according to any one of claims 1 to 3, wherein the allergen is one or more environmental antigen(s).

5. A method according to claim 4, wherein the allergen is in its naturally-occurring form.

6. A method according to claim 4, wherein the allergen is a recombinant protein or synthetic peptide.

7. A method according to any one of claims 1 to 6, wherein the allergen is in a purified form.

8. A method according to any one of claims 1 to 6, wherein the allergen is impure or partially purified.

9. A method according to any one of claims 1 to 8, wherein the allergen is a whole allergen molecule.

10. A method according to any one of claims 1 to 8, wherein the allergen is a part thereof comprising one or more epitopes.

11. A method according to any one of claims 1 to 10, wherein the allergen is selected from the group consisting of house dust mite, animal dander, cockroach, pollen, feathers and mould.

12. A method according to claim 11, wherein the animal dander is selected from the group consisting of cat dander, dog dander and bird dander.

13. A method according to claim 11, wherein the pollen is selected from the group consisting of ryegrass pollen and tree pollen.

14. A method according to any one of claims 1 to 13, wherein the cytokines are selected from the group consisting of IL-4, IL-5, IL-10 and IL-13.

15. A method according to any one of claims 1 to 14, which method further comprises the step of skin pricking a subject and administering either a Th-1 or Th-2 specific allergen.

16. A method of predicting the development of an allergic disease in a subject, comprising:
a). skin prick testing said subject with an allergen capable of inducing Th2 cytokine secretion;
b). determining level of local inflammation; and
c). correlating level of local inflammation with the development of an allergic disease.

17. A method according to claim 16, wherein the subject is a mammalian subject.

18. A method according to claim 16 or claim 17, wherein the subject is a dog, a cat, a livestock animal, a primate and a horse.

19. A method according to any one of claims 16 to 18, wherein the subject is a human subject.

20. A method according to claim 19, wherein the human subject is below the age of about 5 years.

21. A method according to claim 19, wherein the human subject is below the age of about 2 years.
